(19) United States
(12) Plant Patent Application Publication
(10) Pub. No.: US 2002/0174464 P1
(43) Pub. Date: Nov. 21, 2002

(54) Grandiflora rose plant named 'Wezlavn'

(76) Inventor: O. L. Weeks, Chino, CA (US)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 10/199,059

(22) Filed: Jul. 22, 2002

Related U.S. Application Data
(63) Continuation of application No. 09/756,736, filed on Jan. 10, 2001.

(51) Int. Cl. A01H 5/00

(52) U.S. Cl. PLT/138

(57) ABSTRACT

A new and distinct variety of Grandiflora rose plant is provided which forms attractive double blossoms that are light to medium lavender in coloration. The plant exhibits a bushy and well balanced growth habit, and good disease resistance. Medium green semi-glossy foliage is formed. The new plant growth commonly assumes a deep burgundy appearance. The new variety is particularly well suited for growing as attractive ornamentation in the landscape such as in parks and gardens. Cut flower production also is possible.

### SUMMARY OF THE INVENTION

The new variety of Rosa hybrida Grandiflora rose plant was created by artificial pollination wherein two parents were crossed which previously had been studied in the hope that they would contribute the desired characteristics. The female parent (i.e., the seed parent) of the new variety was the ‘Sterling Silver’ variety (United States Plant Pat. No. 1,433). The male parent (i e.. the pollen parent) was the ‘Silver Spoon’ variety (non-patented in the United States). The parentage of the new variety can be summarized as follows:

‘Sterling Silver’33 ‘Silver Spoon’.

The seeds resulting from the above pollination were sown and small plants were obtained which were physically and biologically different from each other. Selective study resulted in the identification of a single plant of the new variety.

It was found that the new variety of Grandiflora rose plant of the present invention possesses the following combination of characteristics:

(a) exhibits a bushy and well balanced growth habit,

(b) forms attractive double blossoms that are light to medium lavender in coloration,

(c) forms medium green semi-glossy foliage,

(d) exhibits good disease resistance, and

(e) is particularly well suited for growing as attractive ornamentation.

The new variety well meets the needs of the horticultural industry and can be grown to advantage for cut flower production, as a specimen plant, or in a mass planting.

The new variety has been found to undergo asexual propagation by a number of routes, including budding, grafting, and cuttage. Asexual propagation by the above-mentioned techniques at Wasco, Calif. has shown that the characteristics of the new variety are stable and are strictly transmissible by such asexual propagation from one generation to another.

The new variety has been named the ‘Wezlavn’ variety. The new variety is being marketed under the SILVER STAR trademark.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were three years of age and were observed during the fall while growing outdoors on their own roots at Wasco, Calif. Dimensions in inches are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view obverse;

FIG. 7—illustrates a specimen of an open flower—plan view reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem with foliage;

FIG. 13—illustrates a specimen of a leaf with three—leaflets plan view—under surface;

FIG. 14—illustrates a specimen of a leaf with five leaflets plan view—upper surface;

FIG. 15—illustrates a specimen of a leaf with seven leaflets plan view—upper surface; and

FIG. 16—illustrates a specimen of maturing petals.

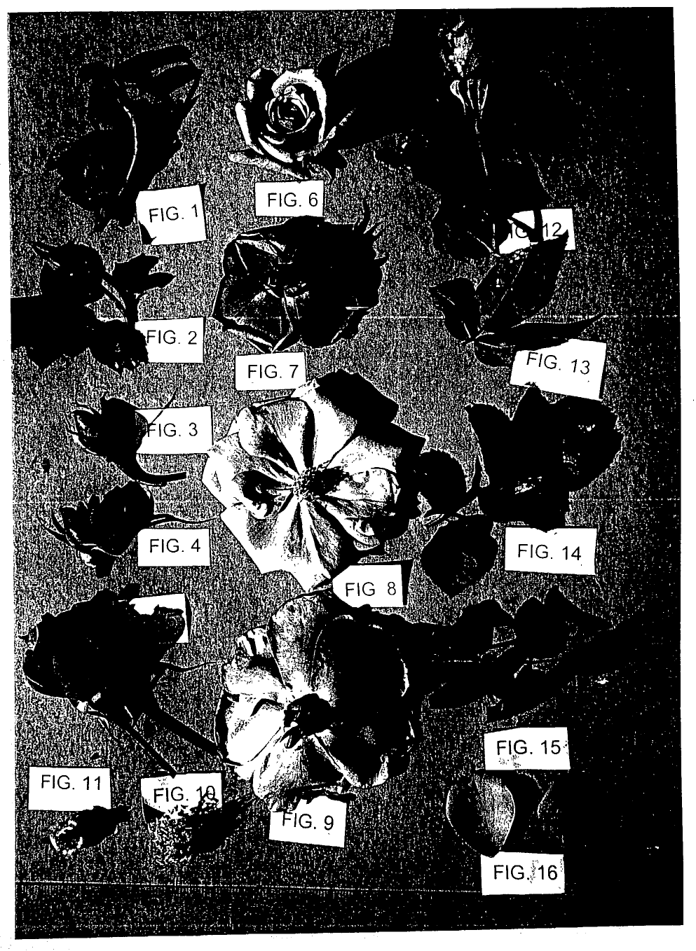

### DETAILED DESRIPTION

The chart used in the identification of the colors is that of The Royal Horticultural Society (R.H.S. Colour Chart). The description is based on the observation of two year-old plants during October while growing on their own roots outdoors at Wasco, Calif. Color terminology is to be accorded its ordinary dictionary significance.

Class: Grandiflora.

Plant:

Height.—approximately 4 to 5 feet at the end of the growing season.

Width.—approximately 2 feet at the end of the growing season.

Habit.—bushy and well balanced.

Branches:

Color.—young stems: Yellow-Green Group 144B with highlights of Greyed-Purple Group 184C. adult wood: Yellow-Green Group 146B.

Thorns.—size: approximately 1 cm in length on average. quantity: moderately numerous, approximately 20 true thorns on a stem having a length of 30 cm. color: near Greyed-Purple Group 184B. shape: needle-shaped.

Leaves:

Stipules.—narrow, triangular, and with auricle facing outwards.

Petioles.—upper surface: Yellow-Green Group 144C. under surface: Yellow-Green Group 144A.

Leaflets.—number: 3, 5, and 7. shape: ovate with serrate margins, rounded base and acuminate apices. serration: regular. texture: consistent. general appearance: medium green with a semi-glossy finish. color (young foliage): upper surface: Green Group 138A with highlights of Red Group 53A. The new plant growth is deep burgundy in coloration. under surface: Green Group 138B with highlights of Greyed-Purple Group 185B. color (adult foliage): upper surface: Green Group 139A with some presence of Green Group 143A. under surface: Green Group 138B.

Infloresence

Number of flowers.—commonly two to five flowers per stem.

Peduncle.—Yellow-Green Group 144B with prickles near Greyed-Purple Group 185B, and the length is approximately 5 to 8 cm on average.

Sepals.—upper surface: near Yellow-Green Group 144A and 144B. under surface: Yellow-Green Group 144A with highlights of Greyed-Purple Group 185B.

Buds.—shape: ovoid. size: large. length: approximately 3 cm on average. color upon opening: upper surface: Red-Purple Group 63A and 63B. under surface: Greyed-Purple Group 186C.

Flower.—shape: double and cup shaped with a flattened center. diameter: approximately 10 cm on average. color (when opening begins): upper surface: Greyed-Purple Group 186C. under surface: Red-Purple Group 63B and 63C. color (when blooming): upper surface: Red-Purple Group 70B and 70C. under surface: Greyed-Purple Group 186C. color (at end of opening): upper surface: Red-Purple Group 69B. under surface: Red-Purple Group 69A with highlights of Red-Purple Group 70D. petal shape: generally heart-shaped. petal number: approximately 30 on average. stamen number: approximately 70 on average. anthers: Greyed-Orange Group 163B in coloration. filaments: Yellow Group 12C in coloration. pistils approximately 35 on average. stigmas: near Yellow-White Group 158B in coloration. styles: near Yellow-Orange Group 11B in coloration. receptacle: separate and free with achenes on the bottom and the wall.

Development

vegetation.—vigorous and strong.

blooming.—substantially continuous throughout the season.

resistance to diseases.—very good.

I claim:

1. A new and distinct variety of Grandiflora rose plant characterized by the following combination of characteristics:
(a) exhibits a bushy and well balanced growth habit,
(b) forms attractive double blossoms that are light to medium lavender in coloration,
(c) forms medium green semi-glossy foliage,
(d) exhibits good disease resistance, and
(e) is particularly well suited for growing as attractive ornamentation; substantially as herein shown and described.
